(19) United States
(12) Patent Application Publication
(10) Pub. No.: US 2017/0143413 A1
(43) Pub. Date: May 25, 2017

(54) Enhanced Safety Method and System for Digital Communication Using Two AC Coupling Wires

(71) Applicant: Biosense Webster (Israel) Ltd., Yokneam (IL)

(72) Inventors: Michael Levin, Haifa (IL); Avi Reuveni, Givat Shmuel (IL)

(21) Appl. No.: 15/340,547

(22) Filed: Nov. 1, 2016

Related U.S. Application Data
(60) Provisional application No. 62/259,370, filed on Nov. 24, 2015.

(51) Int. Cl. A61B 18/14; H04L 27/08; A61B 5/042; H04L 5/14

(52) U.S. Cl. CPC A61B 18/1492; H04L 5/143; H04L 27/08; A61B 5/042; A61B 2017/00053

(57) ABSTRACT

Provided are a method and apparatus for performing medical catheterization using electrical power circuitry disposed outside a medical catheter and remote internal circuitry disposed within the medical catheter. At least one of the power circuitry and the internal circuitry is isolated from electrical ground. Exactly two wires connect the power circuitry to the internal circuitry and a signal generator is provided for generating an alternating carrier that is communicated from the power circuitry to the internal circuitry via the wires. Decoders are disposed in the power circuitry and the internal circuitry, and a transceiver performs half-duplex data communication between the power circuitry and the internal circuitry by alternately modulating the carrier voltage amplitude in one of the power circuitry and the internal circuitry and decoding the modulated carrier voltage amplitude in another of the power circuitry and the internal circuitry.

### CROSS-REFERENCE TO RELATED APPLICATIONS

This Application claims the benefit of U.S. Provisional Application No. 62/259,370, filed 24 Nov. 2015, which is herein incorporated by reference.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD

The presently described subject matter relates to transferring non-mechanical forms of energy to or from the body. More particularly, the described subject matter relates to transferring radiofrequency energy into the heart for ablation.

### BACKGROUND

Mapping of electrical potentials in the heart is now commonly performed, using cardiac catheters comprising electrophysiological sensors for mapping the electrical activity of the heart. Typically, time-varying electrical potentials in the endocardium are sensed and recorded as a function of position inside the heart, and then used to map a local electrogram or local activation time.

When conduction abnormalities, such as atrial fibrillation are present, radiofrequency (RF) ablation of the heart is a procedure that is widely used to correct problematic cardiac conditions. The procedure typically involves insertion of a catheter having an electrode into the heart, and ablating selected regions within the heart with RF energy transmitted via the electrode.

### SUMMARY

Safety requirements can be limiting factors in miniaturization of devices for intra-body applications. For example, catheters used for electrophysiological procedures have various sensors in their distal portions, while processing of the signals from the sensors can occurs proximally. However, providing signal processing capabilities in the distal portion of the catheter can improve the quality of the electrophysiological data and measurements. Signal processing circuitry requires electrical power to be supplied to the distal portion of the catheter. If mechanical failure of the catheter shaft or a short circuit in the power wires supplying the signal processing circuitry were to occur, the subject could experience an electrical shock. The presently described subject matter is directed to methods and systems that provide electrical safety in devices for intra-body applications.

The presently described subject matter is directed to a method, comprising disposing electrical power circuitry outside a medical catheter; disposing remote internal circuitry within the medical catheter; isolating at least one of the power circuitry and the internal circuitry from electrical ground; connecting the power circuitry to the internal circuitry by two wires, for example, by exactly two wires; and communicating an alternating carrier from the power circuitry to the internal circuitry via the two wires. The method may further comprise performing half-duplex data communication between the power circuitry and the internal circuitry by, for example, alternately modulating the carrier voltage amplitude with one of the power circuitry and the internal circuitry and decoding the modulated carrier voltage amplitude with another of the power circuitry and the internal circuitry.

According to one aspect of the presently described method, the power circuitry can comprise a transceiver for modulating the carrier voltage amplitude and a decoder for demodulating the carrier voltage amplitude.

According to a further aspect of the presently described method, the internal circuitry can comprise a decoder for demodulating the carrier voltage amplitude.

Yet another aspect of the method can comprise obtaining and processing data in the internal circuitry from sensors in the catheter and modulating the carrier voltage amplitude according to the processed data for communication a decoder to the power circuitry.

According to still another aspect of the method alternately modulating the carrier voltage amplitude is performed with a first switch in the power circuitry and with a second switch in the internal circuitry to vary first and second resistances across the alternating carrier, respectively.

According to certain embodiments of the presently described subject matter an apparatus, further provided is an apparatus comprising a medical catheter; electrical power circuitry disposed outside the catheter; and remote internal circuitry disposed within the catheter. At least one of the power circuitry and the internal circuitry is isolated from electrical ground. The apparatus may further include two wires, for example, exactly two wires, connecting the power circuitry to the internal circuitry, and a signal generator for generating an alternating carrier that is communicated from the power circuitry to the internal circuitry via the wires. Decoders are disposed in the power circuitry and the internal circuitry, and a transceiver performs half-duplex data communication between the power circuitry and the internal circuitry. The transceiver is operative for alternately modulating the carrier voltage amplitude in one of the power circuitry and the internal circuitry and decoding the modulated carrier voltage amplitude in the decoder of another of the power circuitry and the internal circuitry.

### BRIEF DESCRIPTION OF THE SEVERAL VIEWS OF THE DRAWINGS

For a better understanding of the presently described subject matter, reference is made to the detailed description of the presently described subject matter, by way of example, which is to be read in conjunction with the following drawings, wherein like elements are given like reference numerals, and wherein:

FIG. 1 is a pictorial illustration of a system, which is constructed and operative in accordance with a disclosed embodiment of the presently described subject matter;

FIG. 2 is an electrical schematic of an embodiment of a system for digital communication in accordance with an embodiment of the presently described subject matter; and

FIG. 3 is a flow chart illustrating a sequence of operations using the system shown in FIG. 2 in accordance with an embodiment of the presently described subject matter.

### DETAILED DESCRIPTION

In the following description, numerous specific details are set forth in order to provide a thorough understanding of the various principles of the presently described subject matter. It will be apparent to one skilled in the art, however, that not all these details are necessarily needed for practicing the presently described subject matter. In this instance, well-known circuits, control logic, and the details of computer program instructions for conventional algorithms and processes have not been shown in detail in order not to obscure the general concepts unnecessarily.

Documents incorporated by reference herein are to be considered an integral part of the application except that, to the extent that any terms are defined in these incorporated documents in a manner that conflicts with definitions made explicitly or implicitly in the present specification, only the definitions in the present specification should be considered.

### Overview

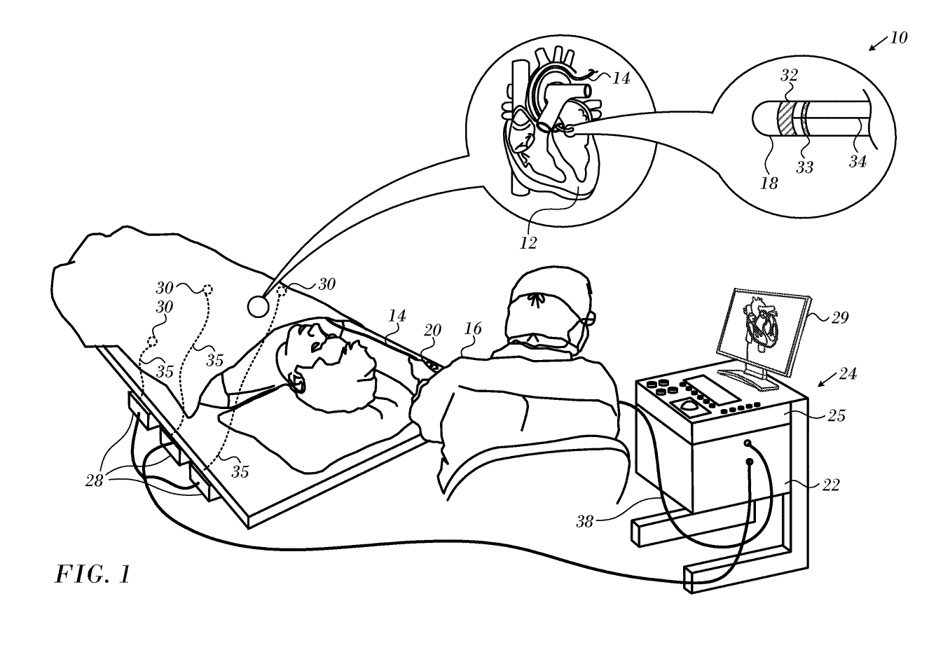

Turning now to the drawings, reference is initially made to FIG. 1, which is a pictorial illustration of a system 10 for evaluating electrical activity and performing ablative procedures on a heart 12 of a living subject, which is constructed and operative in accordance with a disclosed embodiment of the presently described subject matter. The system comprises a catheter 14, which is percutaneously inserted by an operator 16 through the patient's vascular system into a chamber or vascular structure of the heart 12. The operator 16, who is typically a physician, brings the catheter's distal tip 18 into contact with the heart wall, for example, at an ablation target site. Electrical activation maps may be prepared, according to the methods disclosed in U.S. Pat. Nos. 6,226,542, and 6,301,496, and in commonly assigned U.S. Pat. No. 6,892,091, whose disclosures are herein incorporated by reference. One commercial product embodying elements of the system 10 is available as the CARTO® 3 System, available from Biosense Webster, Inc., 3333 Diamond Canyon Road, Diamond Bar, Calif. 91765. This system may be modified by those skilled in the art to embody the principles of the presently described subject matter.

Areas determined to be abnormal, for example by evaluation of the electrical activation maps, can be ablated by application of thermal energy, e.g., by passage of radiofrequency electrical current through wires in the catheter to one or more electrodes at the distal tip 18, which apply the radiofrequency energy to the myocardium. The energy is absorbed in the tissue, heating it to a point (typically about 50° C.) at which it permanently loses its electrical excitability. When successful, this procedure creates non-conducting lesions in the cardiac tissue, which disrupt the abnormal electrical pathway causing the arrhythmia The principles of the presently described subject matter can be applied to different heart chambers to diagnose and treat many different cardiac arrhythmias.

The catheter 14 can comprise a handle 20, having suitable controls on the handle to enable the operator 16 to steer, position and orient the distal end of the catheter as desired for the ablation. To aid the operator 16, the distal portion of the catheter 14 contains position sensors (not shown) that provide signals to a processor 22, located in a console 24. The processor 22 may fulfill several processing functions as described below.

Ablation energy and electrical signals can be conveyed to and from the heart 12 through one or more ablation electrodes 32 located at or near the distal tip 18 via cable 34 to the console 24. Pacing signals and other control signals may be conveyed from the console 24 through the cable 34 and the electrodes 32 to the heart 12. Sensing electrodes 33, also connected to the console 24 are disposed between the ablation electrodes 32 and have connections to the cable 34.

Wire connections 35 link the console 24 with body surface electrodes 30 and other components of a positioning sub-system for measuring location and orientation coordinates of the catheter 14. The processor 22 or another processor (not shown) may be an element of the positioning subsystem. The electrodes 32 and the body surface electrodes 30 may be used to measure tissue impedance at the ablation site as taught in U.S. Pat. No. 7,536,218, issued to Govari et al., which is herein incorporated by reference. A temperature sensor (not shown), including for example, a thermocouple or thermistor, may be mounted on or near each of the electrodes 32.

The console 24 may contain one or more ablation power generators 25. The catheter 14 may be configured to conduct ablative energy to the heart using any known ablation technique, including for example, but not limited to, radiofrequency energy, ultrasound energy, and laser-produced light energy. Such methods are disclosed in commonly assigned U.S. Pat. Nos. 6,814,733, 6,997,924, and 7,156,816, both of which are herein incorporated by reference.

In one embodiment, the positioning subsystem can comprise a magnetic position tracking arrangement that determines the position and orientation of the catheter 14 by generating magnetic fields in a predefined working volume and sensing these fields at the catheter, using field generating coils 28. The positioning subsystem is described in U.S. Pat. No. 7,756,576, which is hereby incorporated by reference, and in the above-noted U.S. Pat. No. 7,536,218.

As noted above, the catheter 14 is coupled to the console 24, which enables the operator 16 to observe and regulate the functions of the catheter 14. Console 24 includes a processor, preferably a computer with appropriate signal processing circuits. The processor is coupled to drive a monitor 29. The signal processing circuits typically receive, amplify, filter, and digitize signals from the catheter 14, including, for example, signals generated by sensors, including but not limited to, electrical, temperature, and contact force sensors, and a plurality of location sensing electrodes (not shown) located distally in the catheter 14. The digitized signals are received and used by the console 24 and the positioning system to compute the position and orientation of the catheter 14, and to analyze the electrical signals from the electrodes.

Typically, the system 10 includes other elements, which are not shown in the figures for the sake of simplicity. For example, the system 10 may include an electrocardiogram (ECG) monitor, coupled to receive signals from one or more body surface electrodes, in order to provide an ECG synchronization signal to the console 24. As mentioned above, the system 10 may also include a reference position sensor, either on an externally-applied reference patch attached to the exterior of the subject's body, or on an internally-placed catheter, which is inserted into the heart 12 maintained in a fixed position relative to the heart 12. Conventional pumps and lines for circulating liquids through the catheter 14 for cooling the ablation site can be provided. The system 10 may receive image data from an external imaging modality, such as an MRI unit or the like and includes image processors that can be incorporated in or invoked by the processor 22 for generating and displaying images.

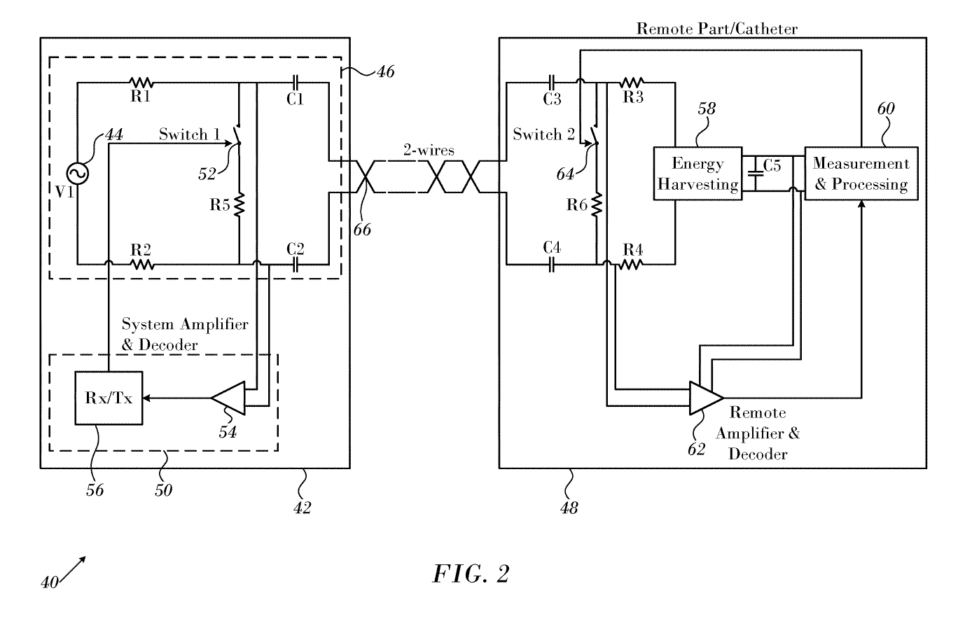

Reference is now made to FIG. 2, which is an electrical schematic of an embodiment of a system for digital communication in a catheter using two alternating current (AC) coupling wires in accordance with an embodiment of the presently described subject matter. The components shown are dimensioned to an intra-body catheter.

A system 40 comprises electrical power circuitry 42, which is can be located outside the catheter, for example, in the console 24 (FIG. 1). Power circuitry 42 comprises an alternating current signal generator 44 connected in a power supply circuit 46. The signal generator 44 generates a carrier frequency in the range of tens or hundreds of kHz. The AC current passes through a network comprising resistors R1, R2 and capacitors C1, C2. The AC current is used both as an electrical energy source for remote circuitry 48 and as the carrier frequency for information transfer between the power circuitry 42 and remote circuitry 48.

Power circuitry 42 includes a signal processing module 50, which controls a switch 52 (ON/OFF) to modulate the carrier frequency. The signal processing module 50 includes an amplifier 54 and a transceiver 56. The amplifier 54 receives and decodes or demodulates signals that are received from internal remote circuitry 48. The transceiver 56 handles communications that are directed to the remote circuitry 48.

The remote circuitry 48 comprises an energy harvesting component 58, a measurement and processing component 60 and an amplifier and decoder 62 for demodulating the carrier voltage amplitude. The energy harvesting component 58, which can be model LTC3331 from Linear Technology, converts the AC voltage at its input to a DC voltage and charges the storage capacitor C5 to a constant value that can be in the range of 3 to 10 volts direct current (VDC). The measurement and processing component 60 and amplifier and decoder 62 are switched in when the DC voltage on the capacitor C5 reaches a predetermined value by switch 64.

As noted above, the remote circuitry 48 is remote from the power circuitry 42. The power circuitry 42 and the remote circuitry 48 are connected by a wire pair 66. The wire pair 66 may be implemented by a twisted pair that reduces sensitivity to external magnetic fields.

### Operation

An AC carrier current produced by signal generator 44 passes through resisters R3, R4 and capacitors C3, C4 in the power circuitry 42; then through wire pair 66 into the remote circuitry 48. Data communication between the power circuitry 42 and remote circuitry 48 is implemented by carrier voltage amplitude modulation. The signal generator 44 together with the resistors R1 and R2 act as the current source and the voltage across the wires of the wire pair 66 depends on the impedance across the two wires.

When both switches 52, 64 are open, i.e., in an OFF state, and the impedance of the capacitors C1 and C2 at the carrier frequency is much less than the values of R1 and R2, the transmission (Tx) voltage between the wires assumes a first value:

Tx=V1*[Rc/(R1+R2+Rc)],

where V1 is the output voltage of the signal generator 44 and Rc is the impedance of the parasitic capacitance and the load of the remote circuitry 48 at the carrier frequency.

As noted above, the signal processing module 50 modulates the carrier voltage amplitude by varying switch 52 between open and closed positions. Signal processing module 50 influences only switch 52 and the remote circuitry 48 influences switch 64. When switch 52 is closed and switch 64 is open, the Tx voltage between across the wire pair 66 assumes a second value:

Tx=V1*(Rc/R1+R2+Rc+R6.

When switch 52 is opened and switch 64 is closed the Tx voltage between across the wire pair 66 assumes a third value:

Tx=V1*(Rc/R1+R2+Rc+R5).

The amplifier and decoder 62 in the remote circuitry 48 receives the modulated carrier voltage and demodulates the information that is embedded in the input signal.

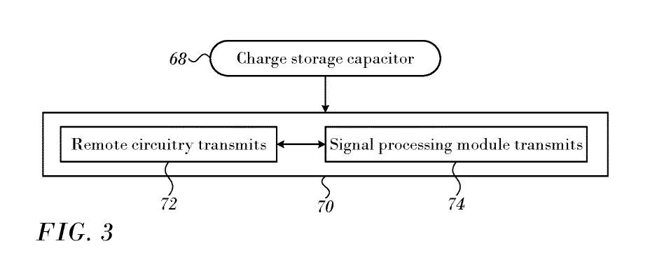

Reference is now made to FIG. 3, which is a flow chart illustrating a sequence of operations using the system 40 (FIG. 2), in accordance with an embodiment of the presently described subject matter. The process steps are shown in a particular linear sequence for clarity of presentation. However, it will be evident that many of them can be performed in parallel, asynchronously, or in different orders. Those skilled in the art will also appreciate that a process could alternatively be represented as a number of interrelated states or events, e.g., in a state diagram. Moreover, not all illustrated process steps may be required to implement the method.

At initial step 68 switches 52, 64 are both opened. The voltage at energy harvesting component 58 is maximal and it charges the storage capacitor C5. When the storage capacitor is charged, the remote unit 48 is ready to work.

Next, communication step 70 is performed, which comprises two steps 72, 74, which are performed in alternation, i.e., the communication is half-duplex. Any suitable communications protocol may be used:

In step 72 the remote circuitry 48 transmits data to the signal processing module 50, modulating a carrier voltage by opening and closing switch 64. Switch 52 remains open during step 72.

In step 74 the signal processing module 50 transmits commands to the remote circuitry 48, modulating the carrier voltage by opening and closing switch 52. Switch 64 remains open during step 74.

It should be noted that the remote circuitry 48 is fully isolated from the power circuitry 42. There is no common ground connection between the two components. If a short between the wires of the wire pair 66 should occur, the patient would be exposed only to a low voltage. Disconnection would result in a higher voltage than normal but still within a low range, so that patient safety would not be compromised. For example, if the generator's output voltage is no more than 2V and resistors R1, R2 are in the range of 50 kΩ, the maximum current through the patient's body would be 2V/100 kΩ=20 uA. In this regard, it may be noted that the maximum allowable current through the patient's body in a single fault condition according to the standard IEC60601-1 is 50 uA.

It will be appreciated by persons skilled in the art that the presently described subject matter is not limited to what has been particularly shown and described hereinabove. Rather, the scope of the present presently described subject matter includes both combinations and sub-combinations of the various features described hereinabove, as well as variations and modifications thereof that are not in the prior art, which would occur to persons skilled in the art upon reading the foregoing description.

1. A method, comprising the steps of:
disposing electrical power circuitry outside a medical catheter;
disposing remote internal circuitry within the medical catheter;
isolating the power circuitry and the internal circuitry from electrical ground;
connecting the power circuitry to the internal circuitry by exactly two wires;
communicating an alternating carrier from the power circuitry to the internal circuitry via the wires, the carrier having a carrier voltage amplitude; and
performing half-duplex data communication between the power circuitry and the internal circuitry by alternately modulating the carrier voltage amplitude with one of the power circuitry and the internal circuitry and decoding the modulated carrier voltage amplitude with another of the power circuitry and the internal circuitry.

2. The method according to claim 1, wherein the power circuitry comprises a transceiver for modulating the carrier voltage amplitude and a decoder for demodulating the carrier voltage amplitude.

3. The method according to claim 1, wherein the internal circuitry comprises a decoder for demodulating the carrier voltage amplitude.

4. The method according to claim 1, further comprising:
in the internal circuitry obtaining and processing data from sensors in the medical catheter; and
modulating the carrier voltage amplitude according to the processed data for communication to the power circuitry.

5. The method according to claim 1, wherein alternately modulating the carrier voltage amplitude is performed with a first switch in the power circuitry and with a second switch in the internal circuitry to vary first and second resistances across the alternating carrier, respectively.

6. An apparatus, comprising:
a medical catheter
electrical power circuitry disposed outside the medical catheter;
remote internal circuitry disposed within the medical catheter, the power circuitry and the internal circuitry being isolated from an electrical ground;
exactly two wires connecting the power circuitry to the internal circuitry;
a signal generator for generating an alternating carrier, the carrier being communicated from the power circuitry to the internal circuitry via the wires, the carrier having a carrier voltage amplitude; and
a respective decoder in the power circuitry and the internal circuitry; and
a transceiver for performing half-duplex data communication between the power circuitry and the internal circuitry, the transceiver operative for alternately modulating the carrier voltage amplitude in one of the power circuitry and the internal circuitry and decoding the modulated carrier voltage amplitude in the decoder of another of the power circuitry and the internal circuitry.

7. The apparatus according to claim 6, wherein the decoder is operative for demodulating the carrier voltage amplitude.

8. The apparatus according to claim 6, further comprising sensors in the catheter, the internal circuitry operative for obtaining and processing data from and modulating the carrier voltage amplitude according to the processed data for communication to the power circuitry.

9. The apparatus according to claim 6, further comprising:
a first switch in the power circuitry and
a second switch in the internal circuitry, the first switch and the second switch operative to vary first and second resistances across the signal generator, respectively.
